(19) United States
(12) Patent Application Publication
(10) Pub. No.: US 2020/0098454 A1
(43) Pub. Date: Mar. 26, 2020

(54) HEALTH NOTIFICATION SYSTEM

(71) Applicant: DAPHNA GALLA, TEANECK, NJ (US)

(72) Inventor: DAPHNA GALLA, TEANECK, NJ (US)

(21) Appl. No.: 16/581,843

(22) Filed: Sep. 25, 2019

Related U.S. Application Data
(60) Provisional application No. 62/736,476, filed on Sep. 26, 2018.

(51) Int. Cl. G16H 10/60; G16H 80/00

(52) U.S. Cl. CPC G16H 10/60; G16H 10/20; G16H 80/00

(57) ABSTRACT

A health notification system and method for providing information about a medical condition or conditions of a child includes an input module for receiving medical information about the child, a storage module for storing the received medical information, an activation module for activating dissemination of the stored medical information to a tagged member caregiver, and an output module for providing the disseminated medical information to the tagged member caregiver. The system and method provide for organized dissemination of the information to multiple caregivers, indefinitely or for pre-determined periods of time.

### CROSS-REFERENCE TO RELATED APPLICATIONS

This application claims the benefit of priority from U.S. Provisional Patent Application No. 62/736,476, filed Sep. 26, 2018.

### BACKGROUND

Food allergies in children appear to be on the rise. At least one in 13 students in any given classroom is likely to be affected by a food allergy, and approximately 1 in 4 will have a reaction while at school. This is a significantly higher number than was the case as recently as 15 years ago. As allergies can be life threatening, prevention plans are recommended in schools and other organizations that are responsible for children. Plans need to be put in place for children with other medical conditions as well (such as asthma, diabetes, epilepsy, etc.).

In general, parents have concerns over the safety of their children in school and feel that additional policies are necessary to improve child safety. Particularly regarding food allergies, food is often ubiquitous throughout the school setting, which creates risks for accidental exposures. Moreover, policies related to field trips and after school activities are lacking.

Currently, parents report health conditions, such as food allergies, to the school nurse or to head camp counselors, for example. This information is gathered, entered into the students'/campers' files, and given out to staff members. Teachers and staff that are responsible for the children must keep track of this critical information in order to ensure their safety at all times. However, there may not be a full-time person on staff who oversees this information. Moreover, there are many staff members in addition to teachers and counselors that need to have this information, such as substitute teachers, specialty staff, lunchroom staff, recess monitors, field trip supervisors, after-school programs, junior counselors, coaches, etc. Compounding the problem is that children and other dependent individuals with allergies often may forget to advise caregivers of an allergy, which can put the child's health in danger.

Even if the information is provided to the relevant parties, it is difficult for the caregivers to keep track of critical information of numerous children in their care, and potentially numerous medical conditions for some of the children. Record keeping is often inaccurate, and critical information cannot easily be accessed on the go. Parents often do not have direct knowledge that the responsible parties have been made aware of the medical condition or conditions of their child.

Existing means to avert unintentional exposures are deficient in many respects. Currently, parents relay the information verbally to caregivers and/or the child wears labels on clothing or bracelets informing others of their allergies. However there is no existing system in place that serves as a backup resource in case parents are not able to relay the information to absolutely every person that the child encounters, or in the event that the child does not want to wear a label or bracelet on their arm.

Thus, there is an urgent need for a system and methods which can improve child health safety in varied environments.

### SUMMARY

There is provided, in accordance with embodiments of the present invention, a system for improvement of health safety for children in a supervised environment. The system includes a first electronic processing device in communication with a software package that is capable of: receiving a health card for a child, wherein the health card includes information about at least one medical condition associated with the child, storing the received health card, and activating dissemination of the health card to a member caregiver. The system also includes a second electronic processing device that is capable of allowing the member caregiver access to the stored health card when said activating occurs.

In accordance with further features of the invention, the medical condition may be an allergy such as a food allergy. In yet additional features of the invention, the health card includes emergency medical instructions for the child related to the medical condition. In some embodiments, the health card includes multiple medical conditions associated with the child, and further includes remedies for each of the multiple medical conditions. In some embodiments, the health card includes explicit step-by-step instructions to the member caregiver of what to do in case of an event related to the medical condition.

In accordance with further features of the invention, the member caregiver may be an organization having multiple supervisors for the child, and the allowed access is automatically provided to all of the multiple supervisors. In additional embodiments, the access is selectively allowed to a pre-defined group or supervisor within the organization. In yet additional embodiments, access is granted for a specific period of time only.

In accordance with yet further features of the invention, the first electronic processing device may also include a notification signal which indicates when one of said member caregivers has accessed the stored health card.

There is provided, in accordance with additional embodiments of the present invention, a system for improvement of health safety for children in a supervised environment. The system includes an input module for receiving medical information about at least one medical condition associated with a child, a storage module for storing the received medical information, an activation module for activating dissemination of the stored medical information to a tagged member caregiver, and an output module for providing the disseminated medical information to the tagged member caregiver.

In accordance with further features of the invention, the received medical information may be a health card including information about the medical condition and instructions for the tagged member caregiver in case of an event related to the medical condition. In some embodiments, the medical condition is an allergy and the medical information includes remedies for an allergic reaction.

In accordance with further features of the invention, the tagged member caregiver may be an organization having multiple supervisors for the child, and the allowed access is automatically provided to all of the multiple supervisors. In other embodiments, the access is selectively allowed to a pre-defined group or supervisor within the organization.

There is provided, in accordance with additional embodiments of the present invention, a method for improvement of health safety for children in a supervised environment. The method includes providing a child health data storage system having taggable caregivers to a user, inputting a health card into the medical data storage system, storing the inputted health card in the child health data storage system until activation is desired, and when desired, activating the stored health card by tagging the member caregiver of the child health data storage system to have access to the health card.

In accordance with further features of the invention, the health card includes personal medical data of a child in the care of the user, and the inputted health card is only accessible to a tagged member caregiver of the child health data storage system. In embodiments, of the invention, the user is a parent of the child.

In accordance with further features of the invention, the health card may include allergy information about the child. In some embodiments, the activating is done for a pre-determined period of time, after which the member caregiver no longer has access to the stored health card. In some embodiments, the member caregiver includes multiple caregivers, and the activating can be done selectively to each of the multiple caregivers at different times. In some embodiments, the member caregiver is an organization which includes multiple individuals responsible for the child, and the method includes ensuring that all of the multiple individuals responsible for the child receive the stored health card. This ensuring may be done by the member caregiver or by the user.

In other aspects of the invention methods and computer program products embodying the invention are provided.

Unless otherwise defined, all technical and scientific terms used herein have the same meaning as commonly understood by one of ordinary skill in the art to which this invention belongs. Although methods and materials similar or equivalent to those described herein can be used in the practice or testing of the embodiments of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Aspects of the invention will be understood and appreciated more fully from the following detailed description taken in conjunction with the appended drawings in which:

FIG. 1 is a simplified conceptual illustration of a system for improvement of health safety for children in a supervised environment, constructed and operative in accordance with embodiments of the present invention;

FIG. 2A is a schematic illustration showing the utility of the system of FIG. 1 from the perspective of a guardian;

FIG. 2B is a schematic illustration showing the utility of the system of FIG. 1 from the perspective of a member caregiver;

FIG. 3 is an illustrative example of a health card of the system of FIG. 1, in accordance with embodiments of the present invention;

FIG. 4 is a schematic illustration of the system of FIG. 1, in accordance with embodiments of the present invention;

FIG. 5 is a flowchart illustration of a method of using the system of FIG. 4, in accordance with embodiments of the present invention; and

FIG. 6 is a simplified block diagram illustration of an exemplary hardware implementation of a computing system, constructed and operative in accordance with an embodiment of the invention.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element.

Further, where considered appropriate, reference numerals may be repeated among the drawings to indicate corresponding or analogous elements. Moreover, some of the blocks depicted in the drawings may be combined into a single function.

### DETAILED DESCRIPTION

The present invention is directed to a system and methods for notification of medical conditions and information to caregivers or supervisors of children. This invention serves as an improved prevention plan to be used by organizations responsible for groups of children and by parents who have children with allergies or other medical conditions.

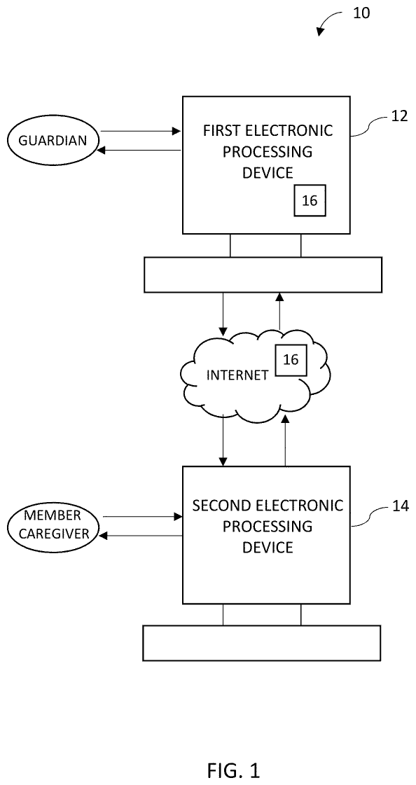

Reference is now made to FIG. 1, which is a simplified conceptual illustration of a system 10 for improvement of health safety for children in a supervised environment, constructed and operative in accordance with embodiments of the present invention. System 10 includes a first electronic processing device 12 and a second electronic processing device 14. First electronic processing device 12 has software and/or hardware which is configured to receive, store and activate a health card 16 for a child, as will be described in greater detail hereinbelow. Second electronic processing device 14 has software and/or hardware which is configured to allow a member caregiver access to the stored health card 16 when the health card is activated via first electronic processing device 12. System 10 is designed to improve health safety for children by allowing a guardian to input relevant health information into first electronic processing device 12 and allowing a member caregiver to retrieve the relevant health information via second electronic processing device 14 when necessary or when desired by the guardian or caregiver. Stored health card 16 may be stored on a central server, on the internet, in the “cloud”, or in any other storage system.

The guardian may be, for example, a parent, grandparent, legal guardian, or any person in charge of the overall welfare of a child. The member caregiver may be, for example, a school, a camp, a sports league, an extra-curricular activity organization, an individual such as a parent in charge of a birthday party, or any other person or group responsible for children when their parents or guardians are not around.

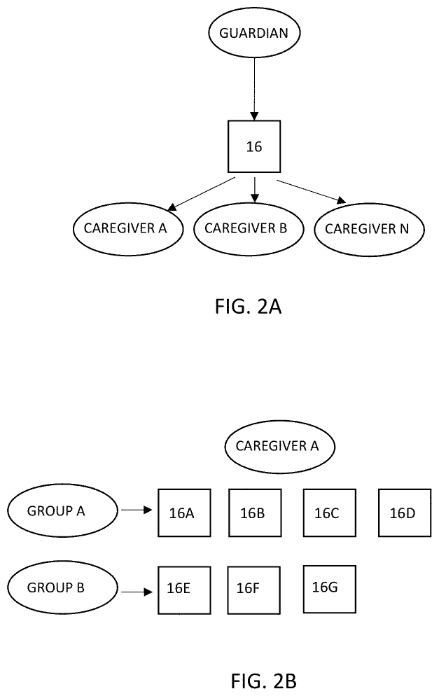

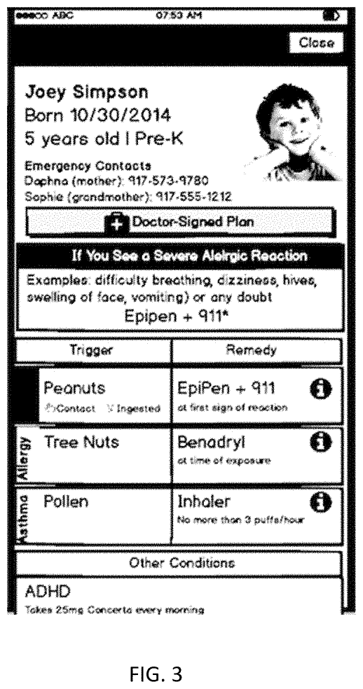

Reference is now made to FIG. 2A, which is a schematic illustration showing the utility of system 10 from the perspective of the parent, and to FIG. 2B, which is a schematic illustration showing the utility of system 10 from the perspective of the member caregiver. A parent or guardian who is responsible for a child with a medical condition such as a food allergy, for example, carries the burden of having to alert all adults that are supervising the child at different times. This is challenging for many reasons. First, the parent or guardian may not always be aware of the totality of supervisors in the child's life. For example, although the parent may have notified the school nurse or even the nurse and the teacher, there may be times when this information does not reach the person in charge at a given time. Some examples may include the lunch staff, a specialty teacher, a substitute teacher, a recess monitor, or others. Moreover, there are circumstances wherein a person is temporarily in charge of the child, such as a parent at a birthday party, or an extracurricular activity, and the parent may not always remember or have the information handy. Even if the parent remembers to tell the caregiver about the condition, the relevant information may not always be provided or clear, and the caregiver may not remember the important facts. Thus, as shown schematically in FIG. 2A, system 10 allows for the parent of a child, designated herein as Child A, to generate a health card 16A specifically for Child A with the relevant information, as will be described hereinbelow with reference to FIG. 3. Health card 16A is then stored in system 10, and can easily be disseminated to as many caregivers (designated as Caregiver A, Caregiver B, Caregiver C. . . etc.) as needed with a simple click of a button: without needing to think about what information to provide each time. Another advantage is that when the parent or guardian updates the medical information, it is automatically updated for each caregiver, which results in decreased concern that out-of-date information is being used.

From the perspective of the caregiver, the caregiver is often responsible for many different children with different medical issues. It is burdensome and time consuming for the staff to enter the medical information, and often inaccuracies occur. Moreover, the information often remains with the nurse or the person in charge at a particular time, and it is burdensome to inform all relevant parties. There also may be particular groups of children—for example, all children in Class 5A, or all children in choir practice, for example. The present system allows for multiple health cards (designated herein as 16A, 16B, 16C, etc.), each one for a different child. These health cards may be organized or classified into groups, for example, so that the relevant information for all children in that group can be seamlessly disseminated to the appropriate supervisors. In addition, instructions about what to do if a child has a medical event or emergency is now available at the click of a button.

Reference is now made to FIG. 3, which is an illustrative example of a health card 16, in accordance with embodiments of the present invention. Health card 16 may include but is not limited to information about the child as follows: name, date of birth and/or age, grade in school, medical condition or conditions, remedies for a medical event, emergency contact information, relationship of emergency contacts to child, and any other details that are desired. In some embodiments, some of the information can be available to certain member caregivers while other or additional information can be available to other member caregivers. For example, if the child has food allergies as well as ADHD, the information given to a parent at a birthday party may be the food allergy information while the information given to a school may also include the ADHD information. In some embodiments, a link to a doctor's plan may be included. In some embodiments, all health cards that include allergy information will include an additional alert, such as a colored note stating: “If you see a severe allergic reaction, please do the following . . . ” In some embodiments, the most relevant information is clearly visible but less critical information may be included via a link or the click of a button.

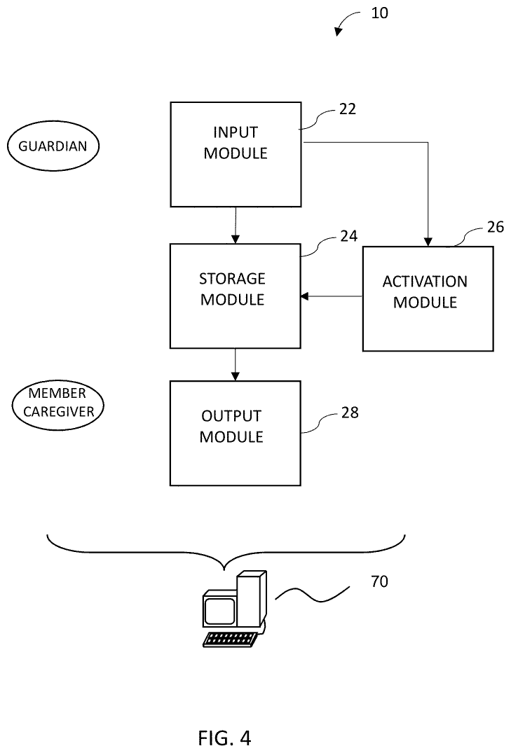

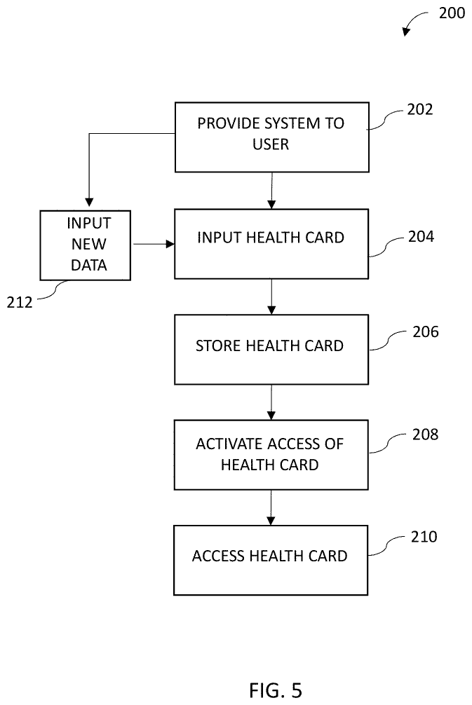

Reference is now made to FIG. 4, which is a schematic illustration of system 10, taken together with FIG. 5, which is a flowchart illustration of a method 200 of using system 10, in accordance with embodiments of the present invention. First system 10 is provided (step 202) to a user, wherein the user is a guardian of a child. Next, the user inputs (step 204) a health card 16 into an input module 22 of system 10, and the inputted health card is stored (step 206) in a storage module 24. Inputting may be done by answering questions on a questionnaire, uploading medical records, uploading other objects such as a picture, providing medical data and remedies, providing specific instructions, providing links to relevant parties, or any other activity which can help complete the health card 16. In some embodiments, an emergency care plan may be input by uploading a picture of the plan, which can be visible via a link or a click. In other embodiments, inputting may include filling in or editing a ready-made template.

Input module 22 is located on or in first electronic processing device 12, such that when the parent or guardian inputs the data for health card 16, the inputting is done on their own personal device such as a smartphone or tablet. Alternatively, inputting may be done on a central processing unit or in any other manner for inputting data. Storing is done in a “cloud” or internet based server, and may also include local storage on first and/or second electronic processing devices 12, 14. Stored health card 16 may be available, and if not activated for a specific period of time may, in some cases, be stored long-term.

When a guardian wants to notify a caregiver of the health condition of the child, the user activates (step 208) access of the health card via an activation module 26. Activation module 26 may be part of first electronic processing device 12, and may be, for example, a physical button or a virtual click, wherein activating activation module 26 causes a particular caregiver to be tagged. In some embodiments, only caregivers who have membership in system 10 can be tagged. Membership can be provided on a short term or long term basis, and activation can be done for any period of time: indefinitely, for a year, months, day, hour, or any relevant time period. In some embodiments, activation module 26 includes choices for who to tag and for how long. For example, in the case of a birthday party, the caregiver (i.e., parent in charge of the party) may generate a code for the duration of the birthday party. The user (guardian, for example) can tag the code such the health card of the child is available to the caregiver for the duration of the party only. Moreover, there may be additional layers of activation, wherein within a caregiver there may be specific groups which can be tagged by the user. In some embodiments, privacy settings may be set differently for different groups. For example, certain medical conditions may only be revealed to particular employees within the member organization.

Once activated, health card is accessed (step 210) from storage module 24 by the tagged caregiver via an output module 28. Output module 28 is located within second electronic processing unit 14, and may include a display, a printout, or any other form of output. In some embodiments, when activation occurs, a notification is sent to the user indicating that the caregiver has accessed the health card. When desired, the user can update information in the health card by inputting new data (step 212) into input module. This new data may then be automatically uploaded to the stored health card so that when the health card is accessed, the new data is present.

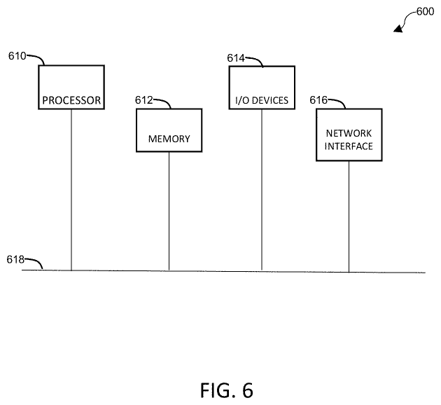

Referring now to FIG. 6, block diagram 600 illustrates an exemplary hardware implementation of a computing system in accordance with which one or more components/methodologies of the invention (e.g., components/methodologies described in the context of FIGS. 1-5) may be implemented, according to an embodiment of the invention. As shown, the invention may be implemented in accordance with a processor 610, a memory 612, I/O devices 614, and a network interface 616, coupled via a computer bus 618 or alternate connection arrangement.

It should be readily apparent that the invention is not limited to the modules disclosed herein, and that other modules may be included as well. The present invention may be used in the form of a website and may downloadable as an application.

Any of the elements shown in FIGS. 1-6 are preferably implemented by one or more computers in computer hardware and/or in computer software embodied in a non-transitory, computer-readable medium in accordance with conventional techniques, such as where any of the elements shown in FIGS. 1, 4 and 5 are hosted by a computer 70.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

Embodiments of the invention may include a system, a method, and/or a computer program product. The computer program product may include a computer readable storage medium (or media) having computer readable program instructions thereon for causing a processor to carry out aspects of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the invention.

Aspects of the invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for improvement of health safety for children in a supervised environment, the system comprising:
a first electronic processing device disposed in communication with a software package that is capable of: receiving a health card for a child, wherein the health card includes information about at least one medical condition associated with the child; storing the received health card; and activating dissemination of the health card to a member caregiver; and
a second electronic processing device that is capable of: allowing the member caregiver access to the stored health card when said activating occurs.

2. The system of claim 1, wherein said at least one medical condition is an allergy.

3. The system of claim 1, wherein said health card comprises emergency medical instructions for the child related to the at least one medical condition.

4. The system of claim 1, wherein said health card includes multiple medical conditions associated with the child, and further includes remedies for each of the multiple medical conditions.

5. The system of claim 1, wherein said health card includes explicit step-by-step instructions to the member caregiver of what to do in case of an event related to the at least one medical condition.

6. The system of claim 1, wherein the member caregiver is an organization having multiple supervisors for the child, and wherein the allowed access is automatically provided to all of the multiple supervisors.

7. The system of claim 1, wherein the member caregiver is an organization having multiple groups or supervisors for the child, wherein the access is selectively allowed to a pre-defined group or supervisor within the organization.

8. The system of claim 1, wherein said first electronic processing device further comprises a notification signal which indicates when one of said member caregivers has accessed the stored health card.

9. The system of claim 1, wherein the access is granted for a specific period of time only.

10. A system for improved health safety for children in a supervised environment, the system comprising:
an input module for receiving medical information about at least one medical condition associated with a child;
a storage module for storing the received medical information;
an activation module for activating dissemination of the stored medical information to a tagged member caregiver; and
an output module for providing the disseminated medical information to the tagged member caregiver.

11. The system of claim 10, wherein the received medical information is a health card including information about the at least one medical condition and instructions for the tagged member caregiver in case of an event related to the at least one medical condition.

12. The system of claim 10, wherein the at least one medical condition is an allergy and the medical information includes remedies for an allergic reaction.

13. The system of claim 10, wherein the tagged member caregiver is an organization having multiple supervisors for the child, and wherein the allowed access is automatically provided to all of the multiple supervisors.

14. The system of claim 10, wherein the tagged member caregiver is an organization having multiple groups or supervisors for the child, wherein the access is selectively allowed to a pre-defined group or supervisor within the organization.

15. A method of improving health safety for children in a supervised environment, the method comprising:
providing a child health data storage system to a user, wherein said child health data storage system has a list of taggable member caregivers, wherein each of said member caregivers is taggable by said user;
the user inputting a health card into the medical data storage system, wherein the health card includes personal medical data of a child in care of the user, and wherein the inputted health card is only accessible to a tagged member caregiver of the child health data storage system;
storing the inputted health card in the child health data storage system until activation is desired;
when desired, activating the stored health card by tagging the member caregiver of the child health data storage system to have access to the health card.

16. The method of claim 15, wherein said user is a parent of the child.

17. The method of claim 15, wherein the health card includes allergy information about the child.

18. The method of claim 15, wherein said activating is done for a pre-determined period of time, after which the member caregiver no longer has access to the stored health card.

19. The method of claim 15, wherein the member caregiver includes multiple caregivers, and wherein the activating can be done selectively to each of the multiple caregivers at different times.

20. The method of claim 15, wherein the member caregiver is an organization the organization comprising multiple individuals responsible for the child, the method further comprising: ensuring that all of said multiple individuals receive the stored health card, wherein said ensuring is done by the member caregiver.
